(19) United States
(12) Patent Application Publication
(10) Pub. No.: US 2004/0215255 A1
(43) Pub. Date: Oct. 28, 2004

(54) Accurate identification of intrinsic atrial activity during overdrive pacing

(76) Inventor: Janneke de Vries, Arnhem (NL)

Correspondence Address: MEDTRONIC, INC., 710 MEDTRONIC PARKWAY NE, MS-LC340, MINNEAPOLIS, MN, 55432-5604, US

(21) Appl. No.: 10/424,579

(22) Filed: Apr. 25, 2003

(51) Int. Cl. A61N 1/362

(52) U.S. Cl. 607/9

(57) ABSTRACT

In general, the invention is directed to the determination of an intrinsic heart rate based on detection of two or more consecutive intrinsic atrial activities to more accurately apply an overdrive pacing therapy in response to pathological events, such as premature atrial contractions (PACs). In this manner, the invention can more accurately identify intrinsic atrial activity as pathological or physiological events, and thus more accurately avoid undue increases in a pacing rate of the overdriving pacing therapy in response to pathological events.

### FIELD OF THE INVENTION

The invention relates to implantable medical devices and, more particularly to cardiac pacemakers that deliver an overdrive pacing therapy.

### BACKGROUND OF THE INVENTION

Tachyarrhythmias are episodes of high-rate cardiac activity. Tachyarrhythmias may occur in one chamber of the heart or may be propagated from one chamber to another. Some tachyarrhythmias are sufficiently high in rate to compromise cardiac output from the chamber affected, leading to loss of consciousness or death in the case of ventricular fibrillation, or weakness and dizziness in the case of atrial fibrillation. Atrial fibrillation is often debilitating, due to the loss of atrial contribution to the cardiac output, and may sometimes lead to ventricular fibrillation.

Fibrillation may be terminated by administering high energy level cardioversion or defibrillation shocks until the fibrillation is terminated. For example, an implanted device may deliver defibrillation shocks via an electrode carried by a lead implanted within the heart. Unfortunately, the high energy levels associated with cardioversion/defibrillation shocks can cause significant pain to the patient. In addition, atrial defibrillation shocks can sometimes give rise to ventricular arrhythmias. Therefore, it is generally desirable to avoid the onset of atrial fibrillation, and thereby avoid the need to apply defibrillation shocks.

Some implanted devices deliver anti-tachycardia pacing pulses to terminate detected episodes of atrial tachycardia. Other devices are configured to continually apply an overdrive pacing therapy, which specifies the delivery of pacing pulses at a rate slightly above an intrinsic heart rate, to maintain control of the heart and reduce a number of atrial tachycardia episodes. In particular, a device can be configured to sense atrial activity due to pacing and intrinsic atrial activity and determine an intrinsic heart rate based on the paced and sensed atrial activities. The device then applies the overdrive pacing therapy at a rate based on the sensed heart rate. By applying overdrive pacing therapy, the device maintains control over the atrium of the heart and provides stable and consistent conduction pathways and refractory periods within the atrium. In this manner, overdrive pacing reduces the incidence of atrial tachyarrhythmias, and avoids the need to deliver painful cardioversion/defibrillation shocks to the patient.

Occasionally, a premature atrial contraction (PAC) with a long coupling interval will occur within the heart and the device will incorrectly detect the “late” PAC as an intrinsic atrial event. The device responds to the PAC as if the intrinsic heart rate increased, and thus increases the rate at which pacing pulses are delivered, i.e., a pacing interval, so that the device can maintain control over the atrium of the heart. However, PACs are generally short disruptions within the heart, and should not be mistaken for intrinsic events. Yet, in some instances, the device may continually identify these late PACs incorrectly as physiological intrinsic events, and respond by increasing the pacing rate. Unfortunately, the pacing rate can quickly accelerate, creating discomfort for the patient, especially in situations where the heart rate should be near rest levels, such as while sleeping. Typically, the solution to this problem is to reduce the maximum rate at which the device can deliver pacing pulses, thereby relieving patient discomfort during the misidentification of PACs. An overdrive pacing therapy with a reduced maximum pacing rate has reduced efficiency, however, because the lower maximum pacing rate narrows the range of rates over which the device can control the heart. Consequently, the device may not be able to respond adequately to increased physiological demands of the patient, e.g., during exercise or other vigorous activity.

### BRIEF SUMMARY OF THE INVENTION

In general, the invention is directed to the determination of an intrinsic heart rate based on detection of two or more consecutive intrinsic atrial activities to more accurately apply an overdrive pacing therapy in response to pathological events, such as premature atrial contractions (PACs). In this manner, the invention can more accurately identify intrinsic atrial activity as pathological or physiological events, and avoid undue increases in a pacing rate of the overdriving pacing therapy in response to pathological events. In particular, the invention can avoid erroneous interpretation of PACS with long coupling intervals, i.e., late PACs, as physiological intrinsic events. Instead, the invention requires the presence of two or more intrinsic events in order to trigger an increase in overdrive pacing.

Notably, an implantable medical device (IMD) determines the intrinsic heart rate based on a sensed intrinsic atrial activity and a subsequently sensed intrinsic atrial activity as opposed to a single sensed atrial activity in response to a pacing pulse and a sensed intrinsic atrial activity. By basing the intrinsic heart rate on two consecutively sensed intrinsic atrial activities, the IMD can correctly distinguish pathological events and in particular PACs from physiological events.

Generally, PACs comprise a single intrinsic atrial activity, which are not followed closely by subsequent intrinsic atrial activities. Physiological events differ from PACs because physiological events comprise two or more intrinsic atrial activities occurring consecutively. Using the intrinsic heart rate as a distinguishing characteristic, the IMD can identify intrinsic atrial activity and increase the pacing rate of the overdrive pacing therapy accordingly. The more accurate identification of the intrinsic atrial activity leads to an overdrive pacing therapy that more accurately correlates to an intrinsic heart rate of a patient, thus increasing patient comfort. In addition, this approach can be effective in conserving limited battery resources within the IMD by avoiding undue delivery of overdrive pacing therapy.

In one embodiment, the invention provides a method comprising sensing an intrinsic atrial activity and a subsequent intrinsic atrial activity within a heart, determining an intrinsic heart rate based on the sensed intrinsic atrial activity and the subsequently sensed atrial activity and delivering an overdrive pacing therapy to the heart based on the intrinsic heart rate.

In another embodiment, the invention provides a device comprising at least one electrode to sense an intrinsic atrial activity and a subsequent intrinsic atrial activity within a heart, and a processor that determines an intrinsic heart rate based on the sensed intrinsic atrial activity and the subsequently sensed atrial activity, and delivers an overdrive pacing therapy based on the intrinsic heart rate.

In another embodiment, the invention is directed to a computer-readable medium containing instructions. The instructions cause a programmable processor to sense an intrinsic atrial activity and a subsequent intrinsic atrial activity within a heart, determine an intrinsic heart rate based on the sensed intrinsic atrial activity and the subsequently sensed atrial activity, and deliver an overdrive pacing therapy to the heart based on the intrinsic heart rate.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic view of an exemplary implantable medical device implanted within a human body.

FIG. 2 is a diagram of the implantable medical device of FIG. 1 located in and near a heart.

FIG. 3 is a block diagram illustrating the constituent components of the implantable medical device depicted in FIGS. 1 and 2.

FIG. 4 is a flow chart illustrating an exemplary process to accurately determine an intrinsic heart rate.

FIG. 5 is another flow chart illustrating an exemplary process to increasing a pacing rate of an overdrive pacing therapy based on accurate identification of a sensed intrinsic atrial activity.

FIG. 6 is a graph illustrating a signal showing a sequence of atrial activities occurring within an atrium of a heart.

FIG. 7 is another graph illustrating a signal showing a sequence of atrial activities occurring within an atrium of a heart.

### DETAILED DESCRIPTION OF THE INVENTION

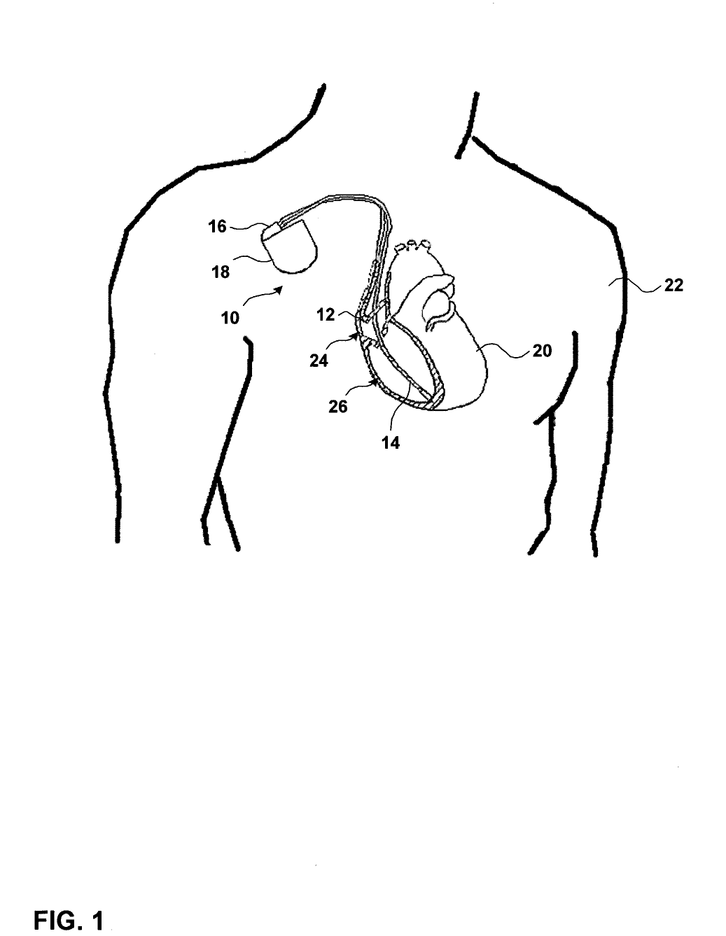

FIG. 1 is a schematic view of an exemplary implantable medical device 10 implanted within a human patient 22. For purposes of illustration, this disclosure refers extensively to the determination of an intrinsic heart rate based on detection of two or more consecutive intrinsic atrial activities to more accurately apply an overdrive pacing therapy in response to premature atrial contractions (PACs). In some embodiments, however, the invention may be applicable to determination of intrinsic heart rates based on detection of two or more consecutive intrinsic atrial activities for purposes other than accurate delivery of overdrive pacing therapies in response to PACs, such as general improved efficiency when delivering the overdrive pacing therapy. Accordingly, a description of the determination of an intrinsic heart rate based on detection of two or more intrinsic atrial activities to accurately classify PACs discussed within this disclosure should not be considered limiting of the invention as broadly claimed and embodied herein.

IMD 10 senses intrinsic atrial activities occurring within heart 20 of patient 22 and accurately determines an intrinsic heart rate. In particular, IMD 10 is capable of accurately distinguishing between intrinsic heart rate increases resulting from physiological events, e.g., exercise, and intrinsic heart rate increases resulting from pathological events, such as PACs. As will be described, IMD 10 senses an intrinsic atrial activity and a subsequent intrinsic atrial activity, determines an intrinsic heart rate between the activities and delivers an overdrive pacing therapy based on the intrinsic heart rate. In general, the overdrive pacing therapy refers to the delivery of pacing pulses at a pacing rate slightly higher than that of the determined intrinsic heart rate.

IMD 10 classifies the sensed intrinsic atrial activity as either a physiological or pathological event based on the intrinsic heart rate relative to a range of rates. The range of rates can depend on the pacing interval, whereby IMD 10 adds and subtracts pre-defined offsets from the pacing interval to define maximum and minimum rates of the range. In response to the comparison, IMD 10 determines whether to increase the pacing rate of the overdrive pacing therapy.

In the example of FIG. 1, IMD 10 is a pacemaker comprising atrial pacing and sensing lead 12 and ventricular pacing and sensing lead 14 attached to connector module 16 of hermetically sealed enclosure 18 and implanted near human or mammalian heart 20 of patient 22. Pacing and sensing leads 12 and 14 sense electrical signals attendant to the activity and repolarization of the heart 20, and further provide the pacing pulses of the overdrive pacing therapy for causing activity of cardiac tissue in the vicinity of the distal ends thereof. Leads 12 and 14 may have unipolar or bipolar electrodes disposed thereon.

IMD 10 is one example of a device capable of practicing the invention, in that IMD 10 has the capability of detecting two or more consecutive intrinsic atrial activities within the heart, determine the intrinsic heart rate based on the intrinsic atrial activities and deliver the overdrive pacing therapy based on the intrinsic heart rate. IMD 10 can further determine a coupling interval between the sensed intrinsic atrial activity and subsequently sensed intrinsic atrial activity to aid in determining the intrinsic heart rate.

In particular, atrial pacing and sensing lead 12 senses atrial activity in the form of an atrial signal, which can consist of intrinsic atrial signals and atrial signals resulting from a pacing pulse IMD 10 delivered. IMD 10 processes the atrial signals and determines whether the signals are intrinsic, which are also referred to as natural atrial senses. IMD 10 can further determine a coupling interval between two intrinsic atrial signals, a sensed intrinsic atrial signal and a subsequently sensed intrinsic atrial signal, and further determine an intrinsic heart rate based on the coupling interval. If IMD 10 determines that the intrinsic heart rate lies within a range of rates indicative of a physiological event, IMD 10 increases the pacing interval of the overdrive pacing therapy. However, if the intrinsic heart rate does not lie within the range of rates, IMD 10 continues to deliver pacing pulses via lead 12 without increasing the pacing rate of the overdrive pacing therapy.

Ventricular pacing and sensing lead 14 senses activation of right ventricle 26, and can also pace right ventricle 26. IMD 10 is not the only implantable medical device that may practice the invention, however. The invention, alternatively, can be practiced by implantable medical devices that are configured to pace one, three or four chambers of heart 20, and that provide overdrive pacing therapies.

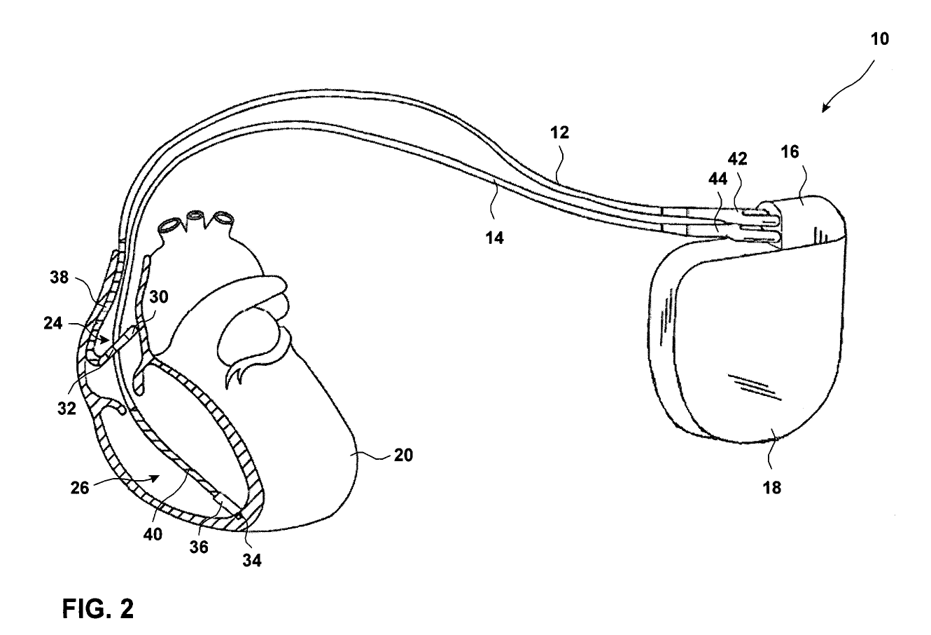

FIG. 2 is a diagram of implantable medical device 10 of FIG. 1 located in and near heart 20. FIG. 2 shows IMD 10, with connector module 16 and hermetically sealed enclosure 18. Atrial and ventricular pacing leads 12 and 14 extend from connector module 16 to the right atrium 24 and right ventricle 26, respectively, of heart 20. Atrial electrodes 30 and 32 disposed at the distal end of atrial pacing lead 12 are located in right atrium 24. Ventricular electrodes 34 and 36 disposed at the distal end of ventricular pacing lead 14 are located in right ventricle 26.

A pulse generator (not shown in FIG. 2) inside enclosure 18 generates pacing pulses for an overdrive pacing therapy. The pacing pulses are delivered to right atrium 24 or right ventricle 26 by electrodes 30, 32, 34, 36. In accordance with the invention, a pacing pulse is delivered to right atrium 24 and two or more atrial signals are also sensed. A processor (not shown in FIG. 2) in IMD 10 determines first whether the atrial signals are intrinsic and second a range of rates. If an intrinsic heart rate, determined by IMD 10 using two or more consecutively sensed intrinsic atrial signals, lies within the range of rates, the sensed intrinsic atrial signal signifies an increase in a pacing rate of an overdrive pacing therapy. By determining the intrinsic heart rate on the basis of two consecutively sensed intrinsic atrial activities, the processor more accurately determines intrinsic heart rate increases, correctly identifies the sensed intrinsic atrial activity and eliminates misidentification of PACs as physiological events, which thereby leads to a more effective overdrive pacing therapy and increased patient comfort.

In particular, PACs are no longer misidentified as physiological events because IMD 10 determines whether the PACs are physiological or pathological events based on the interval between two or more consecutively sensed intrinsic atrial activities. Using two or more consecutively sensed intrinsic atrial activities provides a better indication of the intrinsic heart rate. As a result, the pacing rate of the overdrive pacing therapy no longer increases to a maximum pacing rate in response to PACs, thus increasing patient comfort.

In addition to pacing, IMD 10 can apply other forms of therapy. In FIG. 2, for example, atrial lead 12 and ventricular lead 14 include defibrillation electrodes 38 and 40, respectively. Defibrillation electrodes 38 and 40 deliver defibrillation shocks to right atrium 24 or right ventricle 26 when necessary to terminate an episode of atrial or ventricular defibrillation. Atrial and ventricular leads 12, 14 each include an elongated insulative lead body carrying one or more conductors insulatively separated from one another. At the proximal end of leads 12, 14 are bifurcated connectors 42, 44, which electrically couple the connectors to connector module 16 of IMD 10.

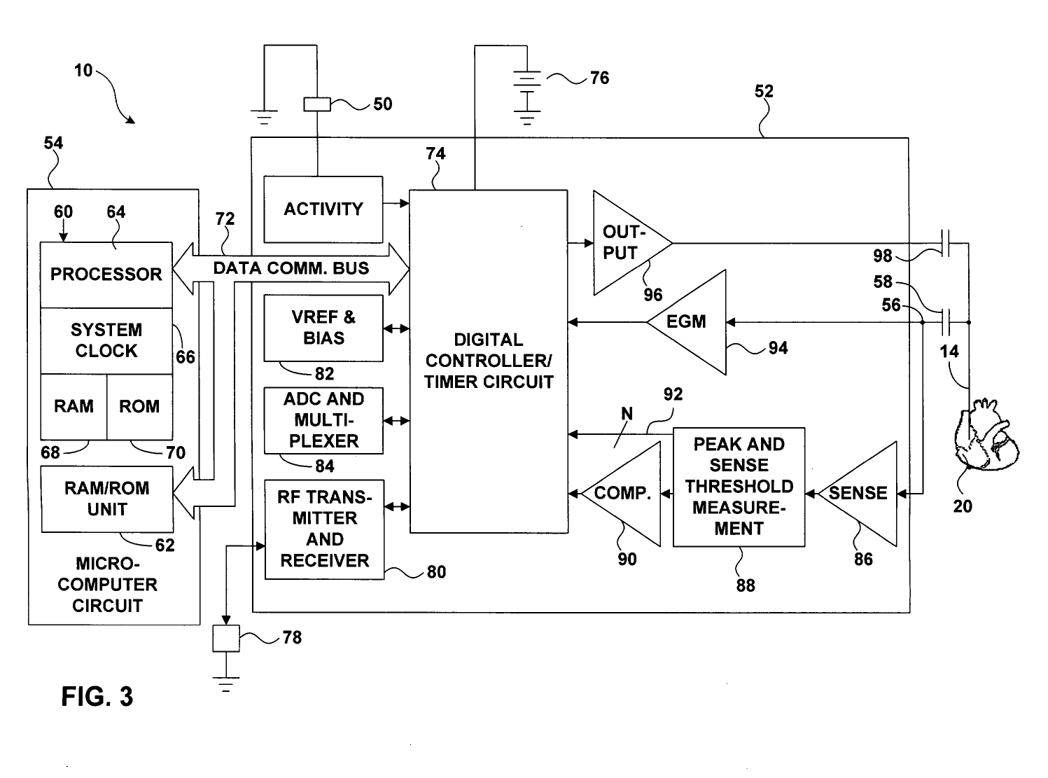

FIG. 3 shows a block diagram illustrating exemplary components of IMD 10 in accordance with one embodiment of the invention, in which IMD 10 comprises a pacemaker having a microprocessor-based architecture. As shown in FIG. 3, IMD 10 can include one or more activity sensors 50. Activity sensor 50 may include an accelerometer, such as a piezoceramic accelerometer or a microelectromechanical accelerometer, that provides a sensor output that varies as a function of a measured parameter relating to a patient's metabolic requirements. In other words, activity sensor 50 detects motion of patient 22 that accompanies physical activity, and can adjust a pacing rate to the metabolic needs associated with the detected physical activity.

The output of activity sensor 50 is coupled to input/output circuit 52. Input/output circuit 52 contains analog circuits for interfacing with heart 20, activity sensor 50, and other components and circuits for the application of stimulating pulses to heart 20. For ease of illustration, IMD 10 in FIG. 3 is shown with only lead 14 connected. Similar circuitry and connections not explicitly shown in FIG. 3 apply to lead 12 (shown in FIGS. 1 and 2), however. Lead 14 is coupled to node 56 in IMD 10 through input capacitor 58.

The rate of heart 20 is controlled by software-implemented algorithms stored within microcomputer circuit 54. In the example of FIG. 3, microcomputer circuit 54 comprises on-board circuit 60 and off-board circuit 62. On-board circuit 60 may include processor 64, system clock circuit 66 and on-board random access memory (RAM) 68 and read-only memory (ROM) 70. Processor 64 may take the form of a microprocessor, digital signal processor (DSP), ASIC, FPGA, or other integrated or discrete logic circuitry capable of performing the functions described herein. Off-board circuit 62 comprises a RAM/ROM unit. On-board circuit 60 and off-board circuit 62 are each coupled by data communication bus 72 to digital controller/timer circuit 74. Microcomputer circuit 54 may comprise a custom integrated circuit device augmented by standard RAM/ROM components.

Microcomputer circuit 54 detects an intrinsic atrial activities and a subsequent intrinsic atrial activity to determine an intrinsic heart rate more accurately and thereby more accurately determining whether the intrinsic atrial activity is a physiological event, such as expected during sleeping, or a pathological event, such as a PAC. If microcomputer circuit 54 identifies the intrinsic atrial activity as a physiological event, a pacing interval of an overdrive pacing therapy is increased to maintain control of an atrium of heart 20. However, if microcomputer circuit 54 identifies the intrinsic atrial activity as a pathological event, the pacing interval of the overdrive pacing therapy is not increased. In accordance with the invention, IMD 10 more accurately determines an intrinsic heart rate based on the intrinsic atrial activity and a subsequent intrinsic atrial activity to prevent misidentification of PACs as physiological events. This more accurate identification process yields improved patient comfort, since the pacing interval of the overdrive pacing therapy more accurately correlates to the actual intrinsic heart rate of a patient, i.e., the pacing rate is near rest levels when the patient is resting.

Processor 64 of IMD 10 compares the determined intrinsic heart rate relative to a range of rates. The range of rates comprises a maximum rate and a minimum rate, both of which processor 64 can calculate from the pacing rate of the overdrive pacing therapy. If the intrinsic heart rate is greater than or equal to the minimum rate of the range and less than or equal to the maximum rate of the range then processor 64 identifies an intrinsic atrial activity corresponding to the intrinsic heart rate as a physiological event and controls digital controller/timer circuit 74 to increase delivery of subsequent pacing pulses associated with the overdrive pacing therapy. However, if the intrinsic heart rate is less than the minimum rate of the range or greater than the maximum rate of the range, then the pacing rate is not increased. The range of rates is sometimes referred to as a physiological band because of the above identification process.

In general, microcomputer circuit 54 stores the maximum and minimum rates of the range to memory, such as RAM 68. Microcomputer 54 can further update the maximum and minimum rates of the range continually as the pacing rate changes, since the range of rates can be based on the pacing rate of the overdrive pacing therapy, which is continually changing. Other limits can also be stored to memory, such as a maximum pacing rate and a minimum pacing rate. The maximum and minimum pacing rates define a maximum and minimum pacing rate at which IMD 10 delivers pacing pulses corresponding to the overdrive pacing therapy. The maximum and minimum pacing rates can be defined prior to implantation into the patient or during device operation. These pacing rates can be altered to improve patient comfort while providing an overdrive pacing therapy.

Electrical components shown in FIG. 3 are powered by an appropriate implantable battery power source 76. For ease of illustration, the coupling of battery power to the various components of IMD 10 is not shown in FIG. 3.

Antenna 78 is connected to input/output circuit 52 to permit uplink/downlink telemetry through radio frequency (RF) transmitter and receiver telemetry unit 80. IMD 10 in FIG. 3 is programmable by an external programmer (not shown) that communicates with IMD 10 via antenna 78 and RF transmitter and receiver telemetry unit 80.

VREF and Bias circuit 82 generates stable voltage reference and bias currents for analog circuits included in input/output circuit 52. Analog-to-digital converter (ADC) and multiplexer unit 84 digitizes analog signals and voltages to provide “real-time” telemetry intracardiac signals and battery end-of-life (EOL) replacement functions. Operating commands for controlling the timing of IMD 10 are transmitted from processor 64 via data bus 72 to digital controller/timer circuit 74, where digital timers and counters establish the overall escape interval of IMD 10 as well as various refractory, blanking and other timing windows for controlling the operation of peripheral components disposed within input/output circuit 52.

Digital controller/timer circuit 74 is coupled to sensing circuitry, including sense amplifier 86, peak sense and threshold measurement unit 88 and comparator/threshold detector 90. Sense amplifier 86 amplifies electrical cardiac signals sensed via lead 14 and provides an amplified signal to peak sense and threshold measurement circuitry 88, which in turn provides an indication of peak sensed voltages and measured sense amplifier threshold voltages on multiple conductor signal path 92 to digital controller/timer circuit 74. An amplified sense amplifier signal is also provided to comparator/threshold detector 90.

Digital controller/timer circuit 74 is further coupled to electrogram (EGM) amplifier 94 for receiving amplified and processed signals sensed by lead 14. The electrogram signal provided by EGM amplifier 94 is employed, for example, when IMD 10 is being interrogated by an external programmer to transmit a representation of a cardiac analog electrogram. Output pulse generator 96 provides pacing stimuli to heart 20 through coupling capacitor 98 in response to a pacing trigger signal provided by digital controller/timer circuit 74.

IMD 10 can sense P-waves, i.e., atrial activities, and R-waves, i.e. ventricular activities, via lead 12 and lead 14, respectively. The signals then propagate through sense amplifier 86, peak sense and threshold measurement unit 88 and comparator/threshold detector 90. IMD 10 further delivers pacing pulses to the atrium and ventricle via leads 12 and 14, respectively. In this manner, two or more consecutive atrial activities can be sensed and a coupling interval can be determined between the two sensed atrial activities. Furthermore, IMD 10 can further deliver pacing pulses corresponding to the overdrive pacing therapy at an increased pacing rate.

Sense amplifier 86, peak sense and threshold measurement unit 88 and comparator/threshold detector 90 are configured to serve as part of an intrinsic atrial activity detector. In response to detection of an intrinsic atrial activity and a subsequent intrinsic atrial activity and determination of the intrinsic atrial activity as a physiological event, processor 64 directs digital controller/timer circuit 74 to decrease an interval of time between consecutive pacing pulses, thus increasing the pacing rate of the overdrive pacing therapy.

As described herein, IMD 10 can sense an intrinsic atrial activity and a subsequent intrinsic atrial activity, determine a more accurate intrinsic heart rate based on the intrinsic atrial activities and deliver an overdrive pacing therapy that more accurately corresponds to an actual intrinsic heart rate of a patient. IMD 10 can employ various methods to determine the intrinsic heart rate but typically a coupling interval between the sensed intrinsic atrial activity and the subsequently sensed intrinsic atrial activity is determined to aid in determining the intrinsic heart rate. IMD 10 can further utilize the determined intrinsic heart rate to deliver an overdrive pacing therapy, which comprises delivery of pacing pulses to a heart at a pacing rate slightly above that of the intrinsic heart rate. Furthermore, IMD 10 can determine a range of rates with which IMD 10 compares the intrinsic heart rate against to determine whether the pacing rate should be increased. In some embodiments, IMD 10 utilizes some or all of the above steps to more accurately apply the overdrive pacing therapy, which aids in properly classifying PACs and prevent patient discomfort.

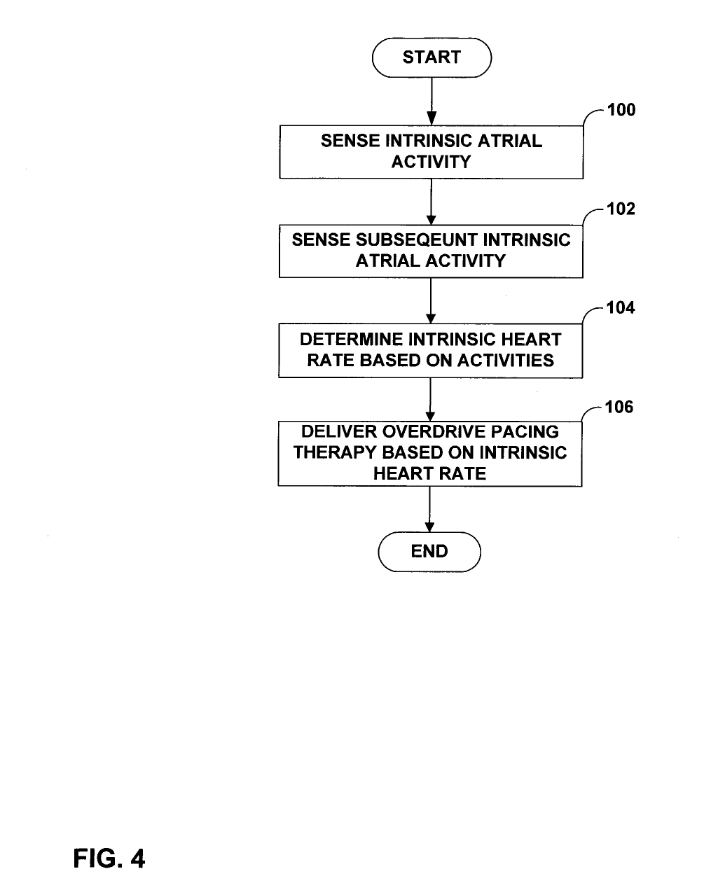

FIG. 4 is a flow chart illustrating an exemplary process to accurately determine an intrinsic heart rate. As shown in FIG. 4, IMD 10 senses an intrinsic atrial activity (100) and a subsequent intrinsic atrial activity (102) via a lead positioned in an atrium of a heart, such as lead 12. Once IMD 10 receives and processes the intrinsic atrial activities, IMD 10 can determine an intrinsic heart rate based on the two consecutive intrinsic atrial activities (104). Via a lead positioned in the atrium of the heart, IMD 10 delivers pacing pulses at a pacing rate slightly above that of the determined intrinsic heart rate, i.e., an overdrive pacing therapy (106).

IMD 10 can determine the intrinsic heart rate from the sensed atrial activities in any manner available. In some embodiments, IMD 10 can determine the intrinsic heart rate from a coupling interval measured between the two sensed intrinsic atrial activities. In some embodiments, IMD 10 can compare the intrinsic heart rate to a range of rates, i.e., the physiological band as described above, and determine or classify the first of two consecutively sensed intrinsic atrial activities as physiological or pathological events. In further embodiments, IMD 10 can specify an overdrive pacing therapy based on the intrinsic heart rate that comprises a pacing rate that continually decreases in slight increments as time elapses. The pacing rate can decrease until a minimum pacing rate is met or until the determined intrinsic heart rate is identified as a physiological event.

Generally, an overdrive pacing therapy responds to a determined intrinsic heart rate that is higher than the pacing rate by increasing the pacing rate to exceed the determined intrinsic heart rate. By increasing the pacing rate, IMD 10 can maintain control over the atrium of the heart, thereby reducing the occurrence of atrial tachyarrhyhthmias. PACs are a type of atrial arrhythmia, which appear as sudden, short increases in the heart rate. IMD 10 identifies the PACs as such and does not increase the pacing rate since the disturbance caused by a PAC is a limited increase indicative of a pathological event.

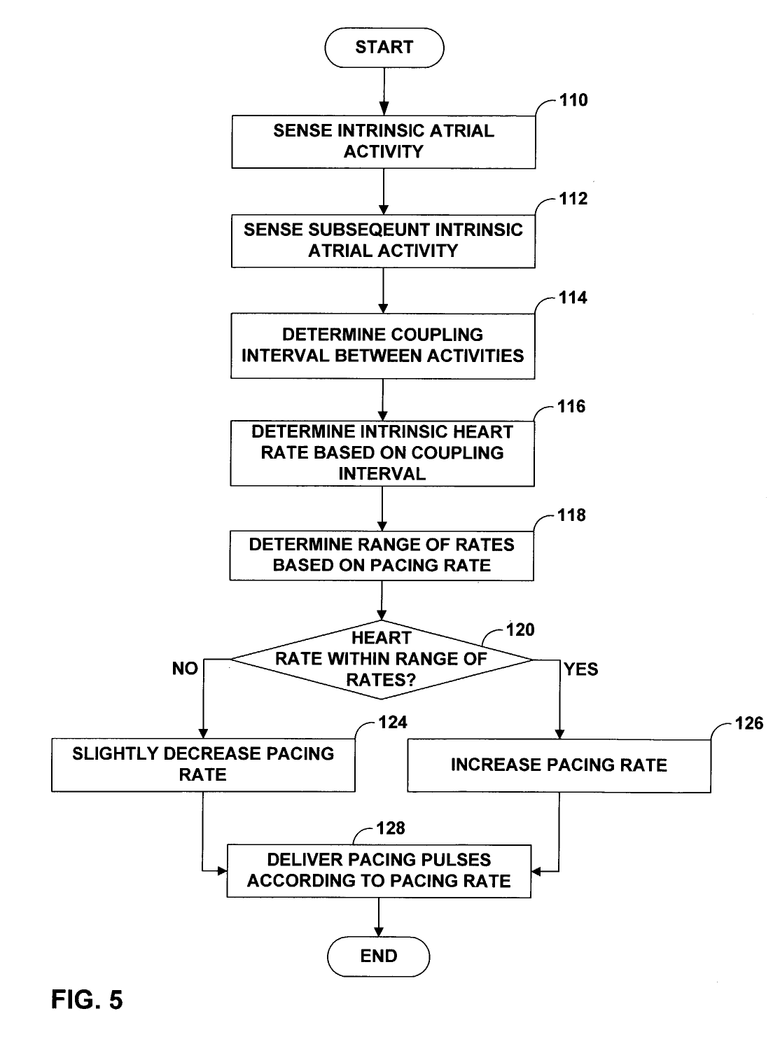

FIG. 5 is another flow chart illustrating an exemplary process to increase a pacing rate of an overdrive pacing therapy based on accurate identification of a sensed intrinsic atrial activity. In particular, the technique determines an intrinsic heart rate based on a coupling interval, which is determined using the sensed atrial activity and a subsequently sensed atrial activity. The intrinsic heart rate is compared against a determined range of rates, i.e., the physiological band, to accurately identify the sensed intrinsic atrial activity.

Before IMD 10 performs the comparison, IMD 10 senses the intrinsic atrial activity (110) and a subsequent intrinsic atrial activity (112). Next, IMD 10 determines a coupling interval between the two consecutively sensed intrinsic atrial activities (114). By measuring the time when each intrinsic atrial activity occurs and subtracting the time when the intrinsic atrial activity occurs from the time when the subsequently sensed atrial activity occurs, IMD 10 can determine the coupling interval. Using two consecutively sensed intrinsic atrial activities IMD 10, can distinguish a pathological PAC from a physiological intrinsic heart rate increase. Since PACs are typically isolated intrinsic activities, the coupling interval between a PAC and a subsequently sensed intrinsic atrial activity can be large. In comparison, an intrinsic heart rate increase representative of a physiological event generally comprises consecutive intrinsic atrial activities, resulting in a small coupling interval.

IMD 10 next determines the intrinsic heart rate based on the coupling interval. The coupling interval defines a time interval between consecutive heart beats, thus by inverting the coupling interval and multiplying by two, IMD 10 can calculate an intrinsic heart rate given a coupling interval (114). When inverting the coupling interval, a small coupling interval leads to a high intrinsic heart rate while a large coupling interval leads to a low heart rate. This relation between the intrinsic heart rate and the coupling interval is notable when determining how to identify a PAC. As described above, PACs typically have a large coupling interval and thus determine a low intrinsic heart rate. True intrinsic heart rate increases have small coupling intervals, which determine high intrinsic heart rates.

The technique stated above allows IMD 10 to accurately calculate the intrinsic heart rate. The intrinsic heart rate of a true intrinsic heart rate increase and that of PACs are shown to contain notable differences, which provide a basis upon which IMD 10 can identify an intrinsic atrial activity. IMD 10 determines a range of rates or physiological band by calculating a maximum rate of the range by incrementing the pacing rate of the overdrive therapy with a set increment and by calculating a minimum rate of the range by decrementing the pacing rate with a set decrement (118). A physician can set both the maximum and minimum rate prior to operation or during operation allowing for a patient-tailored overdrive pacing therapy.

After determining the range of rates, IMD 10 compares the intrinsic heart rate to the determined range of rates (120). If the intrinsic heart rate corresponding to the sensed atrial activity lies within this physiological band, the intrinsic atrial activity is identified as a physiological event or a true intrinsic heart rate increase. If the intrinsic heart rate lies outside the physiological band, the sensed intrinsic atrial activity corresponding to the intrinsic heart rate is identified as a pathological event, such as a PAC. A physiological event indicates to IMD 10 that the heart rate should be higher in response to increased patient activity, patient fear and the like (“YES” branch). IMD 10 will lose control over the atrium of the heart if IMD 10 does not increase the pacing rate of the overdrive pacing therapy. A pathological event signifies a brief lapse in the heart, such as a PAC, and IMD 10 should continue to operate without increasing the pacing rate. (“NO” branch).

A intrinsic heart rate corresponding to a PAC, as discussed above, lies outside the range of rates associated with the pacing rate of the overdrive pacing therapy. Thus, IMD 10 identifies the PAC as pathological and slightly decreases the pacing rate of the overdrive pacing therapy (124). The slight decrease is a feature of some overdrive pacing therapies such that IMD 10 decreases the pacing rate to verify a level of the pacing rate. If the decrease continues for an extended amount of time, this is a method of updating the pacing rate to reflect an intrinsic heart rate of a patient. The decrease can be proportional in magnitude to the pacing rate after a last increase. Thus, variably decreasing rates assures that IMD 10 delivers pacing pulses at a pacing rate slightly above the intrinsic heart rate.

True intrinsic heart rate increases occur as a result of physiological events such as exercise, fear, stress and the like. These are prolonged increases, which can be distinguished from PACs because of their duration. IMD 10 uses two consecutively sensed intrinsic atrial activities to distinguish these types of intrinsic atrial activities. Intrinsic heart rates of physiological events are sustained and comprise intrinsic heart rates within the determined range of rates. IMD 10 increases the pacing interval of the overdrive pacing therapy in response to these physiological events (126) based on the intrinsic heart rate.

In either event, physiological or pathological, IMD 10 continues to deliver pacing pulses according to the newly updated pacing rate (128). IMD 10 further continues to deliver the overdrive pacing therapy until another intrinsic atrial activity is sensed, in which case the process begins to determine whether to increase the pacing rate. Accurate identification of PACs and other anomalies within the heart is crucial to patient comfort. IMD 10 provides an exemplary process to accurately identify physiological events from pathological events. The process specifies numerous calculations, which IMD 10 can carry out in any logical order, some of which are not discussed above but are captured within the scope of the claims below.

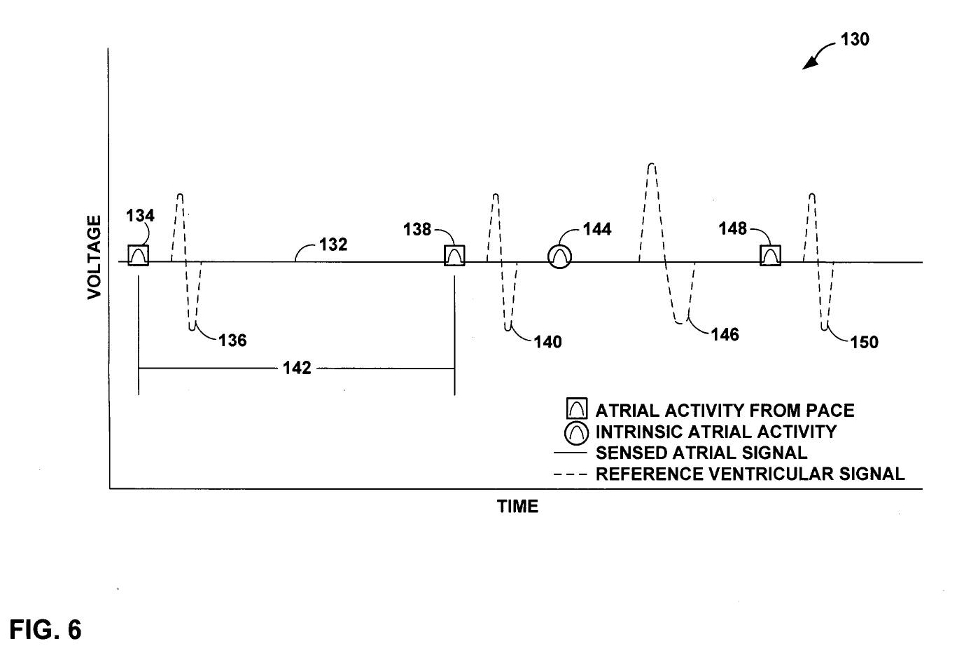

FIG. 6 is a graph illustrating a signal showing a sequence of atrial activities occurring within an atrium of a heart. Graph 130 shows signal 130, which comprises non-intrinsic atrial activities 134, 138, 146 as a result of pacing pulses applied by a medical device, such as IMD 10, and an intrinsic atrial activity 142. Various ventricular activities, i.e., ventricular activities 136, 140, 144, in response to the atrial activities are also represented within graph 130 for reference purposes only. An electrode, such as electrode 12 of FIG. 1, located within the atrium of the heart would not clearly detect ventricular activity.

Graph 130 depicts signal 132 containing non-intrinsic atrial activity 134 resulting from a pacing pulse apart of an overdrive pacing therapy. The medical device delivers the pulse causing the atrium of the heart to contract. Electrical signals corresponding to the contractions are sensed by a medical device, such as IMD 10, via an electrode positioned within the atrium of the heart. Furthermore, the pacing pulse propagates into the ventricles via the Bundle of His and bundle branches to cause the ventricles to contract. Ventricular activity 136 is shown to represent this response of the ventricles to the pacing pulse, although an electrode, such as electrode 12, would not clearly sense these activities as shown.

The next atrial activity along signal 132 is yet another non-intrinsic atrial activity similar to that of non-intrinsic atrial activity 134. Again, ventricular activity 138 is shown to represent ventricular activity in response to non-intrinsic atrial activity 138. Pacing time interval 142 represents an interval of time between non-intrinsic atrial activity 138 and non-intrinsic atrial activity 134. The medical device determines the pacing rate of the overdrive pacing therapy, which indirectly determines time interval 142. Increasing the pacing rate will shorten time interval 142.

Occasionally, an intrinsic atrial activity will occur as depicted within graph 130 by intrinsic atrial activity 144. The medical device senses intrinsic atrial activity 144 and begins the process of identifying intrinsic atrial activity 144. After sensing intrinsic atrial activity 144, the medical device waits to sense a subsequent intrinsic atrial activity.

Intrinsic atrial activity 144 induces ventricular activity 146. The medical device is yet to sense the subsequent intrinsic atrial activity, so the medical device delivers pacing pulses according to the pacing rate of the overdrive pacing therapy, which results in non-intrinsic atrial activity 148 being approximately time interval 142 apart from non-intrinsic atrial activity 138. The pacing pulse also triggers ventricular activity 150. As time elapses to the far right of graph 130, the subsequent intrinsic atrial activity is yet to occur. When the subsequent intrinsic atrial activity does occur, the medical device can calculate a coupling interval between the two consecutively sensed intrinsic atrial activities. The medical device can then determine an intrinsic heart rate based on the coupling interval. After determining a range of rates, i.e., physiological band, based on the pacing rate of the overdrive pacing therapy, the medical device can compare the intrinsic heart rate to the range of rates to identify intrinsic atrial activity 142.

In the case of intrinsic atrial activity 142, the medical device would identify intrinsic atrial activity 142 as a pathological event, which includes a PAC, since the intrinsic heart rate would lie outside of the range of rates. This outcome can be determined by examining graph 130. Since the subsequent intrinsic atrial activity did not occur before non-intrinsic atrial activity 148 or nearly after atrial activity 148, the coupling interval will be large. A large coupling interval determines a low intrinsic heart rate. The low intrinsic heart rate relative to the range of rates, where the minimum rate is typically the pacing rate, will be less than the minimum rate of the range of rates because the coupling interval between the consecutive intrinsic atrial activities is larger than a coupling interval between consecutive non-intrinsic atrial activities. Thus, intrinsic atrial activity is identified as a pathological event.

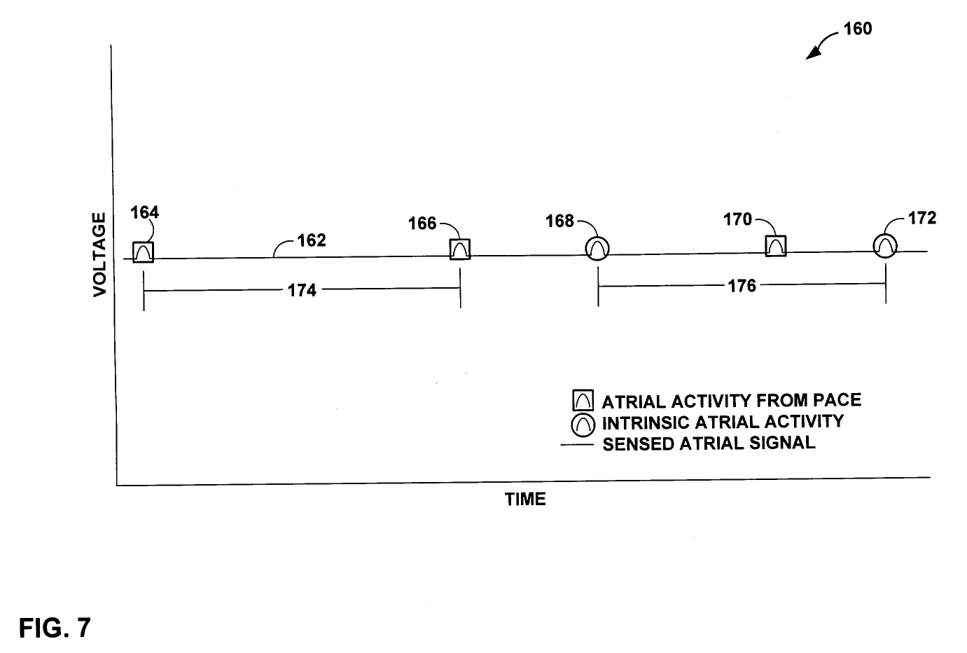

FIG. 7 is another graph illustrating a signal showing a sequence of atrial activities occurring within an atrium of a heart. Graph 160 depicts atrial signal 162 as sensed by a medical device, such as IMD 10 (FIG. 1), via a lead, such as lead 12 having an electrode positioned within an atrium of a heart. The signal comprises non-intrinsic atrial activities 164, 166, 170 resulting from pace pulses delivered by the medical device and intrinsic atrial activities 168, 172. The medical device delivers an overdrive pacing therapy, which comprises delivery of the pacing pulses at a pace rate slightly above that of a determined intrinsic heart rate.

The pace rate is represented indirectly in graph 160 by time interval 174. Increasing the pace rate will shorten time interval 174. After the medical device senses non-intrinsic atrial activities 164, 166, intrinsic atrial activity 168 is sensed. The medical device begins the identification process and waits for subsequent intrinsic atrial activity 168 to occur. Subsequent atrial activity 174 does occur after non-intrinsic atrial activity 170. The medical device calculates coupling interval 176 between intrinsic atrial activity 168 and subsequent intrinsic atrial activity 172. Using the coupling interval, the medical device calculates the intrinsic heart rate. Furthermore, the medical device can calculate a range of rates, also referred to as a physiological band, based on the pacing interval as described above. Comparing the determined intrinsic heart rate to the range of rates, allows the medical device to determine whether or not to increase the pacing rate of the overdrive pacing therapy.

In the case of intrinsic atrial activity 168, coupling interval 176 is less than time interval 174, thus the medical device determines that the intrinsic heart rate is within the range of rates. This can be determined by figuring that a smaller coupling interval or time interval indicates a larger intrinsic heart rate. Applying this principle to coupling interval 176 and time interval 174 yields that the intrinsic heart rate is higher than the pacing rate and that the medical device has lost control over the atrium of the heart. To regain control, the medical device must increase the pacing rate.

Many embodiments of the invention have been described. Various modifications can be made without departing from the scope of the claims. For example, the invention is not limited to the particular techniques described above for determining an intrinsic heart rate. Further techniques can sense a plurality of intrinsic atrial activities, determine a plurality of intrinsic heart rates based on these intrinsic atrial activities and deliver an overdrive pacing therapy based on various combinations of the plurality of intrinsic heart rates. Also, the invention is not limited to the particular implantable medical devices described above, but can be practiced by a wide variety of implantable medical devices. For example, a single chamber implantable medical device can use the invention to more accurately determine an intrinsic heart rate using sensed intrinsic atrial activities. As a result, the single chamber implantable medical device can correctly identify a sensed intrinsic atrial activity while maintaining an efficient overdrive therapy and improving patient comfort.

In addition, the invention may be embodied as a computer-readable medium that includes instructions for causing a programmable processor to carry out the methods described above. A “computer-readable medium” includes, but is not limited to, read-only memory, Flash memory and a magnetic or optical storage medium. The instructions may be implemented as one or more software modules, which may be executed by themselves or in combination with other software.

These and other embodiments are within the scope of the following claims.

What is claimed is:

1. A method comprising:
sensing an intrinsic atrial activity and a subsequent intrinsic atrial activity within a heart;
determining an intrinsic heart rate based on the sensed intrinsic atrial activity and the subsequently sensed atrial activity; and
delivering an overdrive pacing therapy to the heart based on the intrinsic heart rate.

2. The method of claim 1, further comprising determining a coupling interval between the sensed intrinsic atrial activity and the subsequently sensed intrinsic atrial activity to aid in determining the intrinsic heart rate.

3. The method of claim 1, wherein the overdrive pacing therapy comprises delivering pacing pulses at a pacing rate slightly above than the intrinsic heart rate.

4. The method of claim 3, further comprising increasing the pacing rate based on the intrinsic heart rate.

5. The method of claim 4, wherein increasing the pacing rate depends on whether the intrinsic heart rate lies within a range of rates.

6. The method of claim 3, wherein the range of rates comprises a range of rates, where a maximum rate of the range is a set increment of the pacing rate and a minimum rate of the range is a set decrement of the pacing rate.

7. The method of claim 5, wherein the increase is an initial increase and the pacing rate decreases after the initial increase in a manner proportional to the magnitude of the pacing rate after the initial increase.

8. The method of claim 2, wherein the pacing rate increases to a maximum pacing rate limit, which comprises a maximum pacing rate above which the pacing rate cannot increase.

9. The method of claim 7, wherein the pacing rate decreases continually to a minimum pacing rate limit, which comprises a minimum pacing rate below which the pacing rate cannot decrease.

10. The method of claim 1, wherein the intrinsic atrial activity comprises a premature atrial contraction.

11. The method of claim 1, further comprising sensing additional intrinsic atrial activities, determining additional coupling intervals between two consecutive additional intrinsic atrial activities and determining the intrinsic heart rate based on a plurality of additional coupling intervals.

12. A device comprising:
at least one electrode to sense an intrinsic atrial activity and a subsequent intrinsic atrial activity within a heart; and
a processor that determines an intrinsic heart rate based on the sensed intrinsic atrial activity and the subsequently sensed atrial activity and delivers an overdrive pacing therapy based on the intrinsic heart rate.

13. The device of claim 12, wherein the processor further determines a coupling interval between the sensed intrinsic atrial activity and the subsequently sensed intrinsic atrial activity to aid in determining the intrinsic heart rate.

14. The device of claim 12, wherein the overdrive pacing therapy comprises delivering pacing pulses at a pacing rate slightly above the intrinsic heart rate.

15. The device of claim 14, wherein the electrode comprises an electrode to deliver the pacing pulses.

16. The device of claim 14, wherein the processor further increases the pacing rate based on the intrinsic heart rate.

17. The device of claim 16, wherein the processor further determines a range of rates and increases the pacing rate based on whether the intrinsic heart lies within the range of rates.

18. The device of claim 17, wherein the processor further determines a maximum rate of the range by incrementing the pacing rate by a set increment and a minimum rate of the range by decrementing the pacing interval by a set decrement.

19. The device of claim 16, wherein the increase is an initial increase and the processor further decreases the pacing rate after the initial increase in a manner proportional to the magnitude of the pacing rate after the initial increase.

20. The device of claim 16, wherein the processor further increases the pacing rate up to the maximum pacing rate limit, which comprises the maximum pacing rate above which the processor cannot increase the pacing rate.

21. The device of claim 19, wherein the processor further decreases the pacing rate continually until the pacing rate reaches the minimum pacing rate limit, which comprises the minimum pacing rate below which the processor cannot decrease the pacing rate.

22. The device of claim 20, further comprising a memory to store the maximum pacing rate, coupling interval, intrinsic heart rate, and characteristics of the sensed intrinsic atrial activity and subsequently sensed intrinsic atrial activity.

23. The device of claim 13, wherein the intrinsic atrial activity comprises a premature atrial contraction.

24. The device of claim 12, wherein the electrode senses additional intrinsic atrial activities and the processor determines additional coupling intervals between two consecutive additional sensed intrinsic atrial activities and the intrinsic heart rate based on a plurality of additional coupling intervals.

25. A computer-readable medium comprising instructions to cause a processor to:
sense an intrinsic atrial activity and a subsequent intrinsic atrial activity within a heart;
determine an intrinsic heart rate based on the sensed intrinsic atrial activity and the subsequently sensed atrial activity; and
deliver an overdrive pacing therapy to the heart based on the intrinsic heart rate.

26. The computer-readable medium of claim 25, further comprising instructions to cause the processor to determine a coupling interval between the sensed intrinsic atrial activity and the subsequently sensed intrinsic atrial activity to aid in determining the intrinsic heart rate.

27. The computer-readable medium of claim 25, wherein the overdrive pacing therapy comprises delivering pacing pulses at a pacing rate slightly greater than the intrinsic heart rate.

28. The computer-readable medium of claim 27, further comprising instructions to cause the processor to increase the pacing rate based on the intrinsic heart rate.

29. The computer-readable medium of claim 28, further comprising instructions to cause the processor to determine a range of rates based on the pacing rate and increase the pacing rate based on whether the intrinsic heart lies within the range of rates.

30. The computer-readable medium of claim 29, further comprising instruction to cause the processor to determine a maximum rate of the range by incrementing the pacing rate by a set increment and a minimum rate of the range by decrementing the pacing rate by a set decrement.

31. The computer-readable medium of claim 28, wherein the increase is an initial increase and the instructions further cause the processor to decrease the pacing rate after the initial increase in a manner proportional to the magnitude of the pacing rate after the initial increase.

32. The computer-readable medium of claim 28, wherein the instructions further cause the processor to increase the pacing rate to a maximum pacing rate limit, which comprises a maximum pacing rate above which the processor cannot increase the pacing rate.

33. The computer-readable medium of claim 31, wherein the instructions further cause the processor to decrease the pacing rate continually to a minimum pacing rate limit, which comprises a minimum pacing rate above which the processor cannot decrease the pacing rate.

34. The computer-readable medium of claim 25, wherein the intrinsic atrial activity comprises a premature atrial contraction.

35. The computer-readable medium of claim 25, further comprising instructions to cause the processor to sense additional intrinsic atrial activities, determine additional coupling intervals between two consecutive additional intrinsic atrial activities and determine the intrinsic heart rate based on a plurality of additional coupling intervals.
